(19) United States
(12) Patent Application Publication
(10) Pub. No.: US 2015/0320463 A1
(43) Pub. Date: Nov. 12, 2015

(54) Devices for treating bone fractures

(71) Applicant: Ben-Zion Karmon, Petach-Tikva (IL)

(72) Inventor: Ben-Zion Karmon, Petach-Tikva (IL)

(21) Appl. No.: 14/672,181

(22) Filed: Mar. 29, 2015

(30) Foreign Application Priority Data
Mar. 30, 2014 (IL) 231816

(51) Int. Cl. A61B 17/82

(52) U.S. Cl. CPC A61B 17/82

(57) ABSTRACT

A device for treating bone fractures with a sleeve 11 covering the fracture area. The sleeve 11 has inside a stopping element 12 to allow easy filling of the sleeve 11 with bone an easy manipulation. The sleeve 11 can be connected to fixating plate that fixates the bone fragments 15. The stopping element 12 can be fixated to the fixating plate.

This application claims priority from Israeli application No. 231816 filed Mar. 30, 2014.

### FIELD AND BACKGROUND OF THE INVENTION

The present invention relates to an improved methods and devices for treating and healing bone fractures in a living human or animal body.

Non-union of a fracture is the condition in which reparative processes of healing of fracture stops without bone union. Non-union can occur because of mechanical and/or biological circumstances in the environment on the fracture area. Non-union usually requires manipulation and/or bone augmentation to regenerate large quantities of new bone between the bone fragments.

Guided bone regeneration (GBR) membranes are used in dental implant dentistry to regenerate bone. The GBR membranes prevent the epithelial and connective tissue to penetrate the bone defect and interfere with the bone regeneration process. GBR membranes are not commonly used in orthopedics. One reason for not using membranes for treating non-union is because manipulation of a membrane between bone fragments is difficult to perform.

### SUMMARY OF THE INVENTION

The present invention provides a method and device by which to enhance bone regeneration and union of bone fractures. The device is in the shape of sleeve that can be made from a GBR membrane, wherein inside the sleeve there is at least one stopping element that prevents at least part of bone augmenting material to pass through it but allows bone tissue ingrowth and blood vessels penetration through it to regenerate bone inside the sleeve.

The stopping element that allows the ingrowth of bone tissue can be perforated with a large hole or several holes in the size of several hundreds of microns. The sleeve preferably can be filled with a filling material. The filling material preferably includes a bone augmenting material that promotes bone tissue regeneration. The bone augmenting material can include rigid particles that are larger than the perforations in the stopping element. If at least part of the filling material is bio-dissipative then tissue can grow and replace at least part of the filling material resulting in a regeneration of new bone tissue inside the sleeve. The bone augmenting material and the sleeve preferably remain in the body for several weeks or months. The stopping element can be a bio-dissipative material that resorbes faster than the sleeve. The filling material can be suspension of particles or cement or material with high viscosity. Most bone augmenting materials available today are particles or viscous gels or cements or combinations. The size of the holes can be determined by the size of the particles in the bone augmenting material or the degree of the viscosity of the bone augmenting material.

The edges of the sleeve can be placed over the edges of the bone fragments so as to surround the bone fragments and the bone augmenting material being inside the sleeve between the bone fragments. The stopping element can touch one bone fragment and the filling material can touch the other bone fragment or both bone fragments. If there is necrotic bone adjacent the fracture zone, it can be removed and the sleeve with the bone augmenting material is placed in the gap between the bone fragments.

The filling material can be highly viscous, particulated or to become rigid inside the body. The fact that there are holes of several dozens or several hundreds of microns in the stopping element can allow part of the filling material to pass and to touch the bone fragment art the other side of the stopping element and to create one high viscous or rigid substance connecting the bone fragments.

In another embodiment a special bag is used for treating fractures and non-union of small bones like the scaphoid. Fractures of the scaphoid bone are very common and have high incidence of non-union. When treating small bone fractures it can become impractical to remove the necrotic bone from each bone fragment, since the fragments are very small and almost no bone will remain after such removal. In this case a continuous notch is made in the two bone fragments and bone augmentation material is placed in the notch. It will be advantageous to place in the notch a perforated bag filled with bone augmenting material. Preferably the bag has two sides wherein the side of the pouch facing the bone fragments is perforated and the other side facing the surrounding tissues is a GBR membrane that prevents connective tissue and epithelial tissue ingrowth inside the notch along the two bone fragments.

In all embodiments of the present invention a rigid fixation of the bone fragments can be done in addition to the placement of the bag or the sleeve. The rigid fixation can be for example by screws and/or plates.

There are many possible implementations of the devices and methods of the present invention depending on several factors:

1. The place the sleeve is inserted into.

2. The filling material.

3. The shape of the sleeve.

4. The kind of material the sleeve is made of.

The device and method can be therefore used for selective regeneration of more or less specialized tissues, for example, membranes demarcating body cavities and/or separating different tissues and organs from each other, as well as, for selective regeneration of different tissues within the organs, or the organs themselves in relation to the surrounding tissues or nerves. Examples of membranes are the periosteum, the membranes of the brain and the peritoneal membrane; while examples of organs are the bones, bone cavities, liver, the throat, the ventricle, the kidney, the heart and the pancreas. Also, muscle tissue tendons, fat tissue, vessels, ducts, and tubes should be possible to regenerate with this device and method.

Other objects and features of the present invention will become apparent in the following detailed description when taken in connection with the accompanying drawings which disclose one embodiment of the invention. It is to be understood that the drawings are designed for the purpose of illustration only and are not intended as a definition of the limits of the invention.

Thus, according to the teachings of the present invention there is provided a device for treating a bone fracture comprising:

a sleeve having a stopping element, the sleeve being open on a first edge and open on a second edge, the sleeve being made from a guided tissue regeneration membrane, at least part of the stopping element being inside the sleeve, the stopping element being located at least 3 mm from the first edge and at least 3 mm from the second edge.

According to a further feature of the present invention, the sleeve includes holes which are sized to allow the passage of fluids and nutrition and to prevent passage of cells.

According to a further feature of the present invention, the sleeve includes holes having a diameter of less than 5 microns.

According to a further feature of the present invention, the stopping element being perforated with holes having diameter of more than 150 microns.

According to a further feature of the present invention, the sleeve is made at least in part from bio-dissipative material.

According to a further feature of the present invention, the stopping element is made at least in part from bio-dissipative material.

According to a further feature of the present invention, the sleeve is made from collagen.

According to a further feature of the present invention, the sleeve includes a fixating element adjacent at least one of the edges.

According to a further feature of the present invention, the sleeve includes a fixating element selected from the group consisting of a suture, a wire, a band, a screw, a glue, a staple, a tack and any combination thereof. According to a further feature of the present invention, the device further includes a fixating plate and fixating screws.

According to a further feature of the present invention, the device further includes a fixating plate and fixating screws, the stopping element being fixated to the fixating plate.

According to a further feature of the present invention, the stopping element is made from a bone augmenting material.

According to a further feature of the present invention, the sleeve having a first region adjacent the first edge and a second region adjacent the second edge, the regions having a first stiffness and the sleeve between the regions having a second stiffness more than the first stiffness.

According to a further feature of the present invention, the sleeve having a first region adjacent the first edge and a second region adjacent the second edge, the regions having a first diameter and the sleeve between the regions having a second diameter less than the first diameter.

According to a further feature of the present invention, the sleeve having a first region adjacent the first edge and a second region adjacent the second edge, the regions having a first mean time to resorption and the sleeve between the regions having a second mean time to resorption more than the first mean time to resorption.

According to a further feature of the present invention, the sleeve having a first stiffness and the stopping element having a second stiffness more than the first stiffness.

According to a further feature of the present invention, the sleeve having a first mean time to resorption and the stopping element having a second mean time to resorption less than the first mean time to resorption.

According to a further feature of the present invention, the sleeve and the stopping element are made from the same material.

According to a further feature of the present invention, the sleeve is made from two sleeves connected to the stopping element.

Thus, according to the teachings of the present invention there is provided a kit for treating a bone fracture comprising:

a sleeve having a stopping element and a bone augmenting material, the sleeve being open on a first edge and open on a second edge, at least part of the stopping element being inside the sleeve, after the insertion of the bone augmenting material inside the sleeve the stopping element prevents the passage of at least part of the bone augmenting material through the stopping element.

According to a further feature of the present invention, the sleeve is made from a guided bone regeneration membrane.

According to a further feature of the present invention, the sleeve includes holes which are sized to allow the passage of fluids and nutrition and to prevent passage of cells.

According to a further feature of the present invention, the sleeve includes holes having a diameter of less than 5 microns.

According to a further feature of the present invention, the stopping element being perforated with holes having diameter of more than 150 microns.

According to a further feature of the present invention, the sleeve is made at least in part from bio-dissipative material.

According to a further feature of the present invention, the stopping element is made at least in part from bio-dissipative material.

According to a further feature of the present invention, the bone augmenting material includes a bio-active material.

According to a further feature of the present invention, the bone augmenting material includes particles having size of 200-500 microns.

According to a further feature of the present invention, the bone augmenting material includes particles having size of 400-1000 microns.

According to a further feature of the present invention, the bone augmenting material includes particles having size of 900-2500 microns.

According to a further feature of the present invention, the stopping element is made from a bone augmenting material.

According to a further feature of the present invention, the stopping element being located at least 3 mm from the first edge and at least 3 mm from the second edge.

According to a further feature of the present invention, the sleeve is made from collagen.

According to a further feature of the present invention, the sleeve includes a fixating element adjacent at least one of the edges.

According to a further feature of the present invention, the sleeve includes a fixating element selected from the group consisting of a suture, a wire, a band, a screw, a glue, a staple, a tack and any combination thereof.

According to a further feature of the present invention, the kit further includes a fixating plate and fixating screws.

According to a further feature of the present invention, the kit further includes a fixating plate and fixating screws, the stopping element being fixated to the fixating plate.

According to a further feature of the present invention, the bone augmenting material includes a setting material.

According to a further feature of the present invention, the sleeve having a first region adjacent the first edge and a second region adjacent the second edge, the regions having a first stiffness and the sleeve between the regions having a second stiffness more than the first stiffness.

According to a further feature of the present invention, the sleeve having a first region adjacent the first edge and a second region adjacent the second edge, the regions having a first diameter and the sleeve between the regions having a second diameter less than the first diameter.

According to a further feature of the present invention, the sleeve having a first region adjacent the first edge and a second region adjacent the second edge, the regions having a first mean time to resorption and the sleeve between the regions having a second mean time to resorption more than the first mean time to resorption.

According to a further feature of the present invention, the sleeve and the bone augmenting material are made at least partially from a radiolucent material.

According to a further feature of the present invention, the sleeve having a first stiffness and the stopping element having a second stiffness more than the first stiffness.

According to a further feature of the present invention, the sleeve having a first mean time to resorption and the stopping element having a second mean time to resorption less than the first mean time to resorption.

According to a further feature of the present invention, the sleeve and the stopping element are made from the same material.

According to a further feature of the present invention, the stopping element prevents the passage of all the bone augmenting material through the stopping element.

According to a further feature of the present invention, the sleeve is made from two sleeves connected to the stopping element.

Thus, according to the teachings of the present invention there is provided a method for the preparation of a device for treating bone fractures comprising:

Preparing a sleeve from a guided bone regeneration membrane and fixating inside the sleeve a stopping element so the sleeve being open on a first edge and open on a second edge, at least part of the stopping element being located at least 3 mm from the first edge and at least 3 mm from the second edge.

According to a further feature of the present invention, the sleeve is made from two sleeves connected to the stopping element.

According to a further feature of the present invention, the sleeve is made from a guided bone regeneration membrane.

According to a further feature of the present invention, the sleeve includes holes which are sized to allow the passage of fluids and nutrition and to prevent passage of cells.

According to a further feature of the present invention, the sleeve includes holes having a diameter of less than 5 microns.

According to a further feature of the present invention, the stopping element being perforated with holes having diameter of more than 150 microns.

According to a further feature of the present invention, the sleeve is made at least in part from bio-dissipative material.

According to a further feature of the present invention, the stopping element is made at least in part from bio-dissipative material.

According to a further feature of the present invention, the stopping element is made from a bone augmenting material.

According to a further feature of the present invention, the sleeve is made from collagen.

According to a further feature of the present invention, the sleeve includes a fixating element adjacent at least one of the edges.

According to a further feature of the present invention, the sleeve includes a fixating element selected from the group consisting of a suture, a wire, a band, a screw, a glue, a staple, a tack and any combination thereof.

According to a further feature of the present invention, the kit further includes a fixating plate and fixating screws.

According to a further feature of the present invention, the kit further includes a fixating plate and fixating screws, the stopping element being fixated to the fixating plate.

According to a further feature of the present invention, the sleeve having a first region adjacent the first edge and a second region adjacent the second edge, the regions having a first stiffness and the sleeve between the regions having a second stiffness more than the first stiffness.

According to a further feature of the present invention, the sleeve having a first region adjacent the first edge and a second region adjacent the second edge, the regions having a first diameter and the sleeve between the regions having a second diameter less than the first diameter.

According to a further feature of the present invention, the sleeve having a first region adjacent the first edge and a second region adjacent the second edge, the regions having a first mean time to resorption and the sleeve between the regions having a second mean time to resorption more than the first mean time to resorption.

According to a further feature of the present invention, the sleeve having a first stiffness and the stopping element having a second stiffness more than the first stiffness.

According to a further feature of the present invention, the sleeve having a first mean time to resorption and the stopping element having a second mean time to resorption less than the first mean time to resorption.

According to a further feature of the present invention, the sleeve and the stopping element are made from the same material.

Thus, according to the teachings of the present invention there is provided a method for treating bone fractures comprising:

a. inserting a bone augmenting material inside a sleeve, the sleeve being open on a first edge and open on a second edge, the sleeve has at least part of a stopping element inside the sleeve, the stopping element prevents the passage of at least part of the bone augmenting material through the stopping element;

b. placing the first edge around a first bone fragment;

c. placing the second edge around a second bone fragment;

d. fixating the first bone fragment to the second bone fragment so the bone augmenting material and the stopping element being between the first bone fragment and the second bone fragment, the sleeve prevents the penetration of connective tissue inside the sleeve;

e. leaving the sleeve and the bone augmenting material for at least several weeks inside the body to allow the growth of new bone tissue inside the sleeve to connect the first bone fragment with the second bone fragment.

According to a further feature of the present invention, the first bone fragment is in contact with the bone augmenting material and the second bone fragment is in contact the stopping element.

According to a further feature of the present invention, the sleeve is made from a guided bone regeneration membrane.

According to a further feature of the present invention, the sleeve includes holes which are sized to allow the passage of fluids and nutrition and to prevent passage of cells.

According to a further feature of the present invention, the sleeve includes holes having a diameter of less than 5 microns.

According to a further feature of the present invention, the stopping element being perforated with holes having diameter of more than 150 microns.

According to a further feature of the present invention, the sleeve is made at least in part from bio-dissipative material.

According to a further feature of the present invention, the stopping element is made at least in part from bio-dissipative material.

According to a further feature of the present invention, the bone augmenting material includes a bio-active material.

According to a further feature of the present invention, the stopping element being located at least 3 mm from the first edge and at least 3 mm from the second edge.

According to a further feature of the present invention, the sleeve is made from collagen.

According to a further feature of the present invention, the first edge of the sleeve is fixated to the first bone fragment by a first fixating element and the second edge of the sleeve is fixated to the second bone fragment by a second fixating element.

According to a further feature of the present invention, the sleeve includes a fixating element selected from the group consisting of a suture, a wire, a band, a screw, a glue, a staple, a tack and any combination thereof.

According to a further feature of the present invention, the first bone fragment is fixated to the second bone fragment by a fixating plate and fixating screws.

According to a further feature of the present invention, the first bone fragment is fixated to the second bone fragment by a fixating plate and fixating screws the stopping element being fixated to the fixating plate.

According to a further feature of the present invention, the bone augmenting material includes particles having size of 200-500 microns.

According to a further feature of the present invention, the bone augmenting material includes particles having size of 400-1000 microns.

According to a further feature of the present invention, the bone augmenting material includes particles having size of 900-2500 microns.

According to a further feature of the present invention, the stopping element is made from a bone augmenting material.

According to a further feature of the present invention, the bone augmenting material is touching the first bone fragment and the stopping element is touching the second bone fragment.

According to a further feature of the present invention, the bone augmenting material includes a setting material.

According to a further feature of the present invention, the sleeve having a first region adjacent the first edge and a second region adjacent the second edge, the regions having a first stiffness and the sleeve between the regions having a second stiffness more than the first stiffness.

According to a further feature of the present invention, the sleeve having a first region adjacent the first edge and a second region adjacent the second edge, the regions having a first mean time to resorption and the sleeve between the regions having a second mean time to resorption more than the first mean time to resorption.

According to a further feature of the present invention, the sleeve and the bone augmenting material are made at least partially from a radiolucent material.

According to a further feature of the present invention, the sleeve having a first stiffness and the stopping element having a second stiffness more than the first stiffness.

According to a further feature of the present invention, the sleeve having a first mean time to resorption and the stopping element having a second mean time to resorption less than the first mean time to resorption.

According to a further feature of the present invention, the sleeve and the stopping element are made from the same material.

According to a further feature of the present invention, the stopping element prevents the passage of all the bone augmenting material through the stopping element.

According to a further feature of the present invention, the material for promoting the growth of bone includes at least one material selected from the group made up of: an autograft, an allograft, a xenograft, an alloplast, a cytokine, a hormone, a growth factor, a physiologically acceptable drug, a biological modifier, a protein, an antigen, a cell chemotaxis stimulator material, a material inducing osteogenesis, an osteoinduction material, and an osteoconduction material.

According to a further feature of the present invention, the sleeve is formed at least in part from a stretchable material.

According to a further feature of the present invention, the sleeve is formed from more than one type of material.

According to a further feature of the present invention, the sleeve is configured to have a first portion with a first stiffness and a second portion with a second stiffness differing from the first stiffness.

According to a further feature of the present invention, the sleeve is formed at least in part from a material which serves as a selective barrier configured to allow at least a first material to traverse the barrier while preventing passage of at least a second material.

According to a further feature of the present invention, the bone augmenting material includes a self-expanding material.

According to a further feature of the present invention the bone augmenting material is highly viscous.

According to a further feature of the present invention the bone augmenting material includes rigid particles.

According to a further feature of the present invention the sleeve has a first region with a first resistance to passage of a given material and a second region with a second resistance to passage of the given material, the second resistance being less than the first resistance.

According to a further feature of the present invention the given material includes cells and blood vessels.

According to a further feature of the present invention the bone augmenting material includes a bio-active material.

According to a further feature of the present invention the sleeve includes a rigid element.

According to a further feature of the present invention the sleeve being configured after being filled with a setting biocompatible filling material to fixate the tissue as the setting biocompatible filling material sets.

According to a further feature of the present invention the stopping element can be detached from the sleeve and taken out of the sleeve after filling of the sleeve and placing the sleeve between the bone fragments so as to enable direct contact between the bone augmenting material and the two bone fragments.

According to a further feature of the present invention, the stopping element is perforated to allow part of the bone augmenting to pass through the stopping element to allow contact of the bone augmenting with the two bone fragments.

There is also provided according to the teachings of the present invention a device for treating a bone fracture comprising:

a sleeve and a stopping element, said sleeve being open on a first edge to allow the placement of said first edge around a first bone fragment, said sleeve being open on a second edge to allow the placement of said second edge around a second bone fragment to create a space inside said sleeve between said first bone fragment and said second bone fragment, said stopping element being inside said space, said stopping element allows the growth of bone tissue through said stopping element, said stopping element prevents the passage of a bone augmenting material through said stopping element.

There is also provided according to the teachings of the present invention a device for treating a bone fracture comprising:

a sleeve and a stopping element, said sleeve being open on a first edge and open on a second edge, said stopping element being inside said sleeve.

There is also provided according to the teachings of the present invention a method for treating a bone fracture comprising:

a. creating a notch along the bone starting at one bone fragment and continuing to a second bone fracture across the fracture line;

b. filling a bag with a bone augmenting material, the bag has a first side which is perforated with holes having size of more than 100 microns and an opposite side which blocks the penetration of connective and epithelial tissues;

c. placing the bag along the notch so the perforated side of the bag is facing the bone and the opposite side is facing the adjacent tissues.

d. leaving the bag inside the notch for at least several weeks allowing new bone to grow inside the bag to connect the first bone fragment with the second bone fragment.

According to a further feature of the present invention, the bag is made from a guided bone regeneration membrane.

According to a further feature of the present invention, the opposite side of the bag includes holes which are sized to allow the passage of fluids and nutrition and to prevent passage of cells.

According to a further feature of the present invention, the opposite side of the bag includes holes having a diameter of less than 5 microns.

According to a further feature of the present invention, the first side being perforated with holes having diameter of more than 150 microns.

According to a further feature of the present invention, the bag is made at least in part from bio-dissipative material.

According to a further feature of the present invention, the opposite side is made at least in part from bio-dissipative material.

According to a further feature of the present invention, the first side is made at least in part from bio-dissipative material.

According to a further feature of the present invention, the bone augmenting material includes a bio-active material.

According to a further feature of the present invention, the bone augmenting material includes particles having size of 200-500 microns.

According to a further feature of the present invention, the bone augmenting material includes particles having size of 400-1000 microns.

According to a further feature of the present invention, the bone augmenting material includes particles having size of 900-2500 microns.

According to a further feature of the present invention, the bone augmenting material is in the consistency of a viscous gel.

According to a further feature of the present invention, the bag is made from collagen.

According to a further feature of the present invention, the bag includes a fixating element.

According to a further feature of the present invention, the bag includes a fixating element selected from the group consisting of a suture, a wire, a band, a screw, a glue, a staple, a tack and any combination thereof.

According to a further feature of the present invention, the bag further includes a fixating plate and fixating screws.

According to a further feature of the present invention, the bone augmenting material includes a setting material.

According to a further feature of the present invention, the first side having a first stiffness and the opposite side having a second stiffness more than the first stiffness.

According to a further feature of the present invention, the first side having a first mean time to resorption and the opposite side having a second mean time to resorption more than the first mean time to resorption.

According to a further feature of the present invention, the bag and the bone augmenting material are made at least partially from a radiolucent material.

According to a further feature of the present invention, the first side and the opposite side are made from the same material.

According to a further feature of the present invention, the first side prevents the passage of all the bone augmenting material through the stopping element.

### BRIEF DESCRIPTION OF THE DRAWINGS

The invention is herein described, by way of example only, with reference to the accompanying drawings, wherein:

FIG. 1 is a perspective view illustrating the novel device used in accordance with the invention to receive and contain bone augmentation material.

FIG. 2 is a perspective view illustrating the device of FIG. 1 after it was filled with bone augmenting material and placed between two bone fragments.

FIG. 3 is a bottom view illustrating the device of FIG. 2 after fixating the sleeve to the bone fragments and fixating the two bone fragments with a fixating plate and screws.

FIG. 4 is a perspective view illustrating an embodiment of a bag for treating fractures of small bones.

FIG. 5 is a perspective view illustrating a notch created along a broken scaphoid bone.

FIG. 6 is a perspective view of the device of FIG. 3 after placement inside the notch along the broken scaphoid of FIG. 5.

FIG. 7 is a perspective view of the device of FIG. 3 after placement inside the notch along the broken scaphoid of FIG. 5 as illustrated in FIG. 6 in which the bone fragments are fixated to each other by a fixating plate and screws.

### DESCRIPTION OF THE PREFERRED EMBODIMENTS

As mentioned further above there are many implementations of the invention in different tissues and organs. The following description will focus on embodiments for treating bone fractures and non-union of bone fractures. The same principles should be used in other tissues and organs.

Before turning to the features of the present invention in more detail, it will be useful to clarify certain terminology as will be used herein in the description and claims. Specifically, it should be noted that the present invention is useful in a wide range of applications in which living tissue is to be regenerated The term “living tissue” is used herein to refer to any living tissue including, but not limited to bone, an organ, tube, vessel, cavity, bone cavity or membrane, and interfaces between any two or more of the above. Where used within a single type of tissue, the typical application of the present invention is for regenerating the tissue inside the tissue. When used at a tissue interface, the invention is typically used to separate between tissues.

In another matter of terminology, it is noted that a large number of different types of materials are known which may be inserted within the body during a surgical procedure and which later dissipate, thereby avoiding the need for a separate surgical procedure for their removal. Such materials are properly referred to, depending upon the mechanism by which the material dissipates, as “bioresorbable”, “bioabsorbable” or “biodegradable”. Despite the differences between these different classes of materials, the aforementioned terminology is widely used interchangeably by medical professionals. Accordingly, and for conciseness of presentation, only one of these terms will generally be used in the following description, without implying the exclusion of the other classes of materials. Additionally, the phrase “bio-dissipative material” is used herein in the description and claims to refer generically to any and all materials which dissipate without requiring surgical removal, independent of which mechanisms such as dissolution, degradation, absorption and excretion take place. The actual choice of which type of materials to use may readily be made by one ordinarily skilled in the art.

The bone can be regenerated by several biological mechanisms: Osteogenesis in which the bone augmenting material includes bone forming cells; Osteoinduction in which the bone augmenting material includes materials that induce cells to form bone or to differentiate to become bone forming cells; Osteoconduction in which the bone augmenting material provides a scaffold for bone forming cells; or Osteopromotion in which encouraging the biologic or mechanical environment of bone regeneration.

Finally with respect to terminology, reference will be made to a biocompatible filling material used to fill the sleeve of the present invention. It should be noted that this filling material may assume a wide range of compositions and consistencies, so long as the biocompatible material may be inserted into the sleeve. Thus, possible consistencies for the filling material include, but are not limited to, consistencies described as watery, viscous, gelatinous, moldable, waxen, particulate, and suspensions or mixtures combining any of the above.

Turning now in detail to the drawings, which depict the presently preferred embodiments of the invention for the purpose of illustrating the practice thereof and not by way of limitation of the scope of the invention, and in which like reference characters refer to corresponding elements throughout the several views.

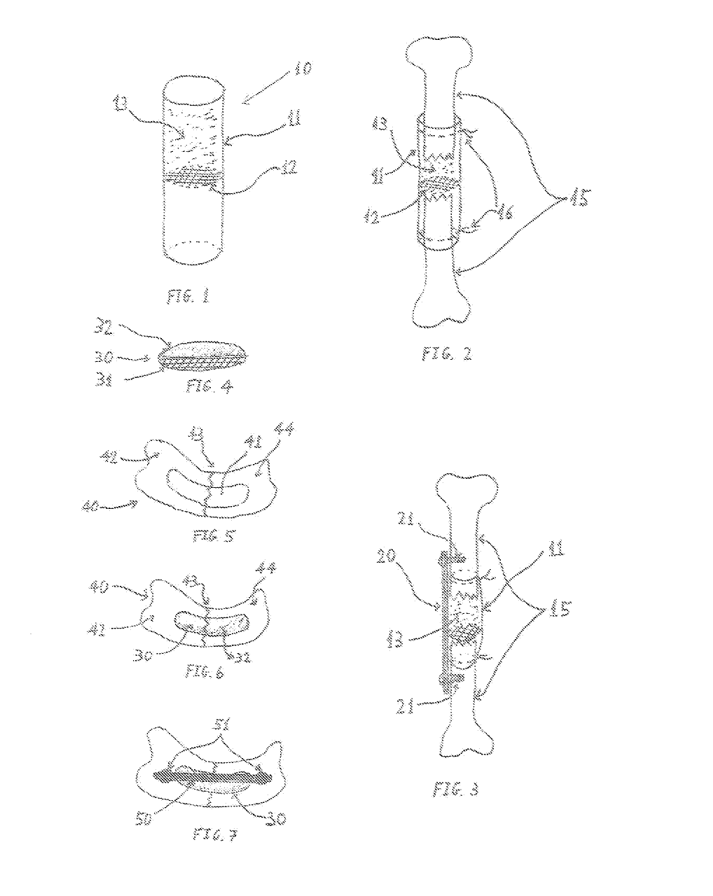

FIG. 1, illustrates an embodiment of the novel device for use in treating bone fractures and/or non-union of bone fractures. The device is in the shape of a sleeve 11 having inside a stopping element 12. The sleeve can be filled with a filling material 13 preferably a bone augmenting material that promotes bone tissue healing and/or regeneration. The stopping element 12 prevents at least part of the filling material 13 from falling outside the sleeve 11. The sleeve 11 can be made from a guided bone regeneration (GBR) membrane. The stopping element 12 can be made from a bone augmenting material. The stopping element 12 can be made also from a GBR membrane and/or a perforated GBR membrane or can be made for example from a bone plate or and/or a perforated bone plate. The stopping element can be made from a mesh that can be flexible. The mesh can be made from resorbable and/or non-resorbable sutures. The mesh can be made from biocompatible material like titanium. The sleeve can be flexible.

FIG. 2 illustrates the use of the device between two bone fragments 15. The sleeve 11 is open on both edges to allow the placement of each edge over a bone fragment 15. The sleeve 11 can include fixating components to fixate the edges of the sleeve to the bone fragments 15. The fixation component can be for example a suture 16 or a wire placed around the sleeve or sutured adjacent the sleeve edges. The sleeve can be also fixated to the bone fragments by other means for example screws and/or tacks and/or staples and/or tacks and/or biocompatible glue. Each of the fixating components can be also bioresorbable.

The use of a sleeve having a stopping element allows easy and efficient filling and compacting the bone augmenting material. If the sleeve is filled without the stopping element the bone augmenting material can get out from the opposite opening of the sleeve and compacting of the bone augmenting material becomes more difficult. The sleeve having a stopping element can be filled easily outside the body and then to be placed between the bone fragments. A GBR membrane can be also rolled around the bone fragments, but this take time and the filling of the bone augmenting material become more difficult. It is also possible to place one edge of the sleeve around the first bone fragment, then to fill the pouch and afterwards to place the second edge around the second bone fragment. For working this way it is advantageous to have a flexible sleeve and the filling can be more complicated and time consuming than filling the sleeve outside the body.

The sleeve can also be attached to a fixating plate 20 as illustrated in FIG. 3, which is used to fixate the bone fragments. The fixating plate 20 can be fixated to the bone fragments 15 for example by screws 21. The fixating plate 20 can be connected to the sleeve 11 before placement of the sleeve 11 between the bone fragments 15 or after the placement of the sleeve 11.

In another embodiment the stopping element 12 can be also fixated to the fixating element, for example by a screw, a pin, a wire, a suture, by glue or any other fixating element. The fixating element can be also bioresorbable. This fixating element, for example the screws, can penetrate the sleeve so it is passing through the fixating plate 20 and the sleeve 11 inside the stopping element 12. In this embodiment the stopping element 12 being stabilized and therefore the sleeve 11 is also more stable. In a preferred embodiment the fixating element of the stopping element penetrate only partially inside the stopping element, for example 1-5 mm or 2-4 mm or 2.5-3.5 mm inside the stopping element, so not to interfere with the bone growth through the stopping element 12.

In a preferred embodiment the sleeve 11, the stopping element 12 and the fixating plate 20 are already assembled together so the stopping element 12 is fixated to the fixating plate 20.

In another embodiment only part of the sleeve 11 is fixated to the fixating plate 20 to allow easy filling of the sleeve 11 after the bone fragments 15 are fixated by the fixating plate 20. The sleeve can be fixated at the stopping element region for example by the fixating screw. The sleeve 11 can be also fixated to the fixating plate 20 on one side of the stopping element 12 and not fixated to the fixating plate 20 on the other side.

In another embodiment the sleeve 11 has a side perforation and preferably two side perforations on each side of the stopping element 12, so bone can be inserted inside the sleeve 11 after the sleeve 11 being placed between and over the bone fragments. If the side perforation is small (diameter of less than 5 mm) the side perforation can be left open. The side perforation preferable being closed by tor example sutures, staples or glue.

The stopping element 12 can be perforated with holes which are large enough to allow bone tissue ingrowth. The holes in the stopping element 12 can be of diameter of 100-2000 microns. The holes can be of diameter of 250-1500 microns. The holes can be of diameter of 500-1000 microns. The sizes of the holes can be compatible with the size of the particles of the bone augmenting material 13 which are inserted inside the sleeve 11.

The sleeve 11 can be filled on both sides with the bone augmenting material 13 or can be filled only from one side as illustrated in FIGS. 2 and 3. The edges of the sleeve 11 can be place around the bone fragment 15 as illustrated in FIGS. 2 and 3 to cover 1-10 mm of the bone fragment 15. The edges of the sleeve 11 can be place around the bone fragment 15 to cover 2-8 mm of the bone fragment 15. The edges of the sleeve 11 can be place around the bone fragment 15 to cover 3-5 mm of the bone fragment 15.

The sleeve 11 and/or the stopping element 12 can be made from autograft, allograft, xenograft and alloplast and any combination thereof.

The sleeve 11 and/or the stopping element 12 can be made from a biocompatible material. The sleeve can be made at least in part from resorbable materials to prevent a chronic foreign body reaction like: conventionally available 15 polyglycolic acid (PGA) mesh, a high-molecular-weight linear polymer made by the ring opening polymerization of the purified glycolide monomer, although other suitable materials might be used e.g. polyglactin 910, i.e. polyglycolide co-galactide and types of ammonio methacrylate copolymer. In addition PDS (another absorbable suture material) or cellulose might possibly also be used as a sleeve material.

The sleeve can be made from collagen. It can be made from variety types of collagen. It can include cross-linked collagen. It can be made also from natural membranes of the body like pericardium of humans or animals.

The device 10 can be made of more than one type of material. The device can be resorbable or not resorbable or partially resorbable. The device can be made from two types of resorbable materials. The stopping element 12 can be made from a material that is rapidly resorbed and allows bone ingrowth and the sleeve 11 can be made from a second type that is slowly resorbed and prevent the ingrowth of epithelial and connective tissues. The stopping element 12 can be perforated allowing contact between the bone augmenting material 13 and the two bone fragments 15. The stopping element can have a region which is perforated. The perforated region of the stopping element can have pores that allow the bone augmenting material to pass through the stopping element 12 to wet the opposite side of the stopping element, but prevents falling of the filling material 13. The size of the pores are dependent on the filling material. For PMMA cement the pore size in the stopping element 12 can be in the range of 0.05-0.5 mm depending on the viscosity of the cement. The perforated region of the stopping element can have one or several macro holes of several dozens of microns or to have one or several big holes of several millimeters. The end result is a continuous rigid mass of cement connected to the bone fragments and going inside the sleeve.

The sleeve and/or the stopping element can be also made from not resorbable materials like ePTFE.

The sleeve 11 and/or the stopping element 12 and or the bone augmenting material 13 can include antibiotics. The sleeve can include also stretchable and/or elastic materials. Materials include, either alone or in combination, metals or metal alloys, polymers, carbon and ceramics. Exemplary metallic members include stainless steel, titanium, tantalum, shape-memory materials such as nickel-titanium alloy (NiTi) (Compounds using NiTi are manufactured under the marks NITINOL™ and ELASTINITE™ and are available from several sources), Elgiloy (trade name) and NP35N (trade designation), which can provide desired degree of springiness, malleability and/or response to temperature changes. Exemplary polymers include polyurethanes, silicon rubbers, polyether, sulfones, fluoroelastomers, polyimides, polycarbonates, polyethylens, polylactic acid, polyglycolic acid, polyacrylates, polyurethanes like polycarbonate urethane and the like and combinations and copolymers thereof which provide a variety of abilities to bioabsorb or biodegrade or to be totally inert. The sleeve can include springs and coils that are compressed before insertion.

In another embodiment the sleeve can include materials with different degree of stiffness. The material touching the bone fragments 15 can be less stiff than the material between the bone fragments.

The combination of several regions with different degree of stiffness can influence the shape of the filled sleeve. The sleeve can have variable shapes and the volumes according to the use. For example to reconstruct the rib the sleeve can be rounded like C-shape.

In another embodiment the sleeve is like a double walled sleeve placed so the bone fragments are inside the sleeve or in the shape of a double walled sheet that can be placed around the bone fragments.

In another embodiment at least part of the stopping element can be taken out after placement of the sleeve between the bone fragments. This can be done for example if the stopping element is made from a folded suture having its end protruding outside the sleeve. After placing the filled bag between the bone fragments the protruding suture end is pulled and taken out of the sleeve leaving a continuous bone augmenting material inside the sleeve between the two bone fragments.

In another embodiment the sleeve has a small cut near the stopping element. The cut can be already in sleeve or made by the surgeon during the procedure. After placing the device between the bone fragments, the stopping element is taken out through this cut. The stopping element can be flexible or easily folded to allow easy withdrawal through the small cut.

The withdrawal of the stopping element can be done before or after fixating the sleeve edges to the bone fragments. The small cut can be sutured back by the surgeon after taking out the stopping element.

The device can be made by rolling a membrane around the stopping element and connecting the membrane borders to create a sleeve. The connection of the membrane's borders can be for example by sutures, staples and/or tacks and/or glues. The sleeve can be made from tubular organs like the intestine, blood vessels and/or the bladder. The stopping element can be done from a more stiff material like a bone plate.

The device can be made also be connecting two sleeves to the stopping element. Each sleeve on the other side of the stopping element, creating a continuous sleeve having a stopping element inside.

The sleeve can be made from collagen and the stopping element from a synthetic material like tricalcium phosphate and/or hydroxyapatite.

In another embodiment the sleeve also includes a selective barrier that permits transfer of some cells and materials and prevents the transfer of other cells and materials. Therefore allowing bone forming cells and blood to get inside the sleeve and block the entrance of connective tissue cells. This barrier can also permit the release of medication mixed with the filling material without letting the filling material to leak. This barrier should be adopted for its specific use for example to have little holes, sized according to the medicine to be released.

In another embodiment the sleeve can be made from a rigid mesh and a bio-dissipative membrane attached to the mesh and closes the holes of the mesh. The advantage of this configuration is that the sleeve can be filled and maintain its shape and prevents connective tissue to enter the space where bone is expected to regenerate, but after the membrane is resorbed blood vessels can enter from the periosteum and/or the adjacent tissues and supply the new bone. The mesh can be perforated like a titanium mesh with pore size of 0.2-1 mm. The sleeve can be made only from the titanium foil without a membrane.

The bone augmenting material can be an autograft, an allograft, a xenograft, an alloplast, a cytokine, a hormone, a growth factor, a physiologically acceptable drug, a biological modifier, a protein (for example Bone Morphogenetic Protein (like BMP-2, BMP-7)), an antigen, a cell chemotaxis stimulator material, a material inducing osteogenesis, an osteoinduction material, an osteoconduction material, a bioactive material, a bioresorbable material, a bioabsorbable material, a biodegradable material and any combination thereof. The bone augmenting material can include materials that occupy a space in the body for at least several months. These materials preferably encourage the tissue to grow inside the space occupied by the filling material. This is the principle function of most bone augmenting materials available on the market. The bone augmenting material can be resorbable. The bone augmenting material can be available in the market like hydroxyapatite, bovine mineral (i.e. Bio-Oss available from Geistlich, Swiss), demineralized frizzed dried bone allograft, synthetic materials like PLA (i.e. FisioGraft from Ghimas) or suspension of bovine mineral in a liquid medium. The bone augmenting material can be also fully or partially not bioresorbable, for example crystal hydroxyapetit.

The bone augmenting material can include therapeutic materials.

The bone augmenting material can be a biocompatible filing material that sets and becomes rigid inside the tissue. After the material has set the bone fragments near the fracture are fixated. The biocompatible filling material can be a bioresorbable material that contains materials assisting in the process of bone healing like bone cements, for example Skeletal Repair System (SRS) from Norian company, Healos from Orquest company, OsteoGenics and Orthovita's Orthocomp from Howmedical Leibinger company.

Most bone augmenting materials are available as particles in the size of 200-2000 microns. To allow easy insertion preferably the particles are mixed with a solution like saline, blood or biocompatible gels like cellulose, glycerol and hydrogel. If the size of the pores in the stopping element are 1.5-5 times greater than the size of the particles of the bone augmenting material the sleeve can be filled with bone augmenting material, so some particles can pass through the stopping element but no massive falling can occur. The pores can be 3 times the particles size. This feature allows filing of the pouch with minor leakage and immediate direct contact between the bone augmenting material and the two bone fragments without falling of the bone augmenting material during manipulation. The sleeve can be also filled using a high viscous gel like Dinagraft which is gelatinous allograft bone augmenting material or with bone cements like PMMA or calcium sulfate or calcium carbonate. The end result is one continuous mass of the filling material which is rigid or highly viscous connecting the two bone fragments inside the sleeve for at least several weeks.

The sleeve 15 itself, can include materials that promote bone tissue growth, for example proteins like BMP and growth factors.

FIG. 4 illustrates another embodiment a device to treat bone fractures. The device can be in the shape of a bag 30 having one side perforated 31 and the other side 32 not perforated. The bag 30 can be filled with the bone augmenting material. This bag 30 can be used for the treatment of fractures of small bones like the scaphoid. FIG. 5 illustrates a broken scaphoid 40. A continues notch 41 is done in the broken bone 40 starting at the first bone fragment 42 and over the fracture line 43 continuing to the second bone fracture 44, as illustrated in FIG. 5. The filled bag 30 is placed inside the notch as illustrated in FIG. 6. The perforated side 31 (not illustrated) is facing the bone surface and the non-perforated side 32 is facing the tissues adjacent the bone. Therefore bone tissue coming from the bone fragments 42, 44 can grow through the perforations in the perforated side 31 and new bone tissue will grow inside the bag 30. The non-perforated side 32 will prevent the surrounding tissues from interfering with the bone tissue growth inside the bag 30. In addition the bone fragments 42, 44 can be fixated with fixating plate 50 and/or fixating screw 51 as illustrated in FIG. 7. The fixating plate 50 can be placed over the bag 30, as illustrated in Fig., to fixate the bag 30 to the notch 41. The fixating plate 50 can be also placed without touching the bag 30.

The bag 30 can be made from the same materials as the sleeve 11 described above. The bone augmenting material filling the bag 30 can include any combination of the materials described above to fill the sleeve 11. As described above the bone augmenting material can be bone cement which will slightly pass through the perforated side 31 and wet external side of the perforated side to connect the two bone fragments 42, 44. The size of the pores in the perforated side can be according to the viscosity of the bone cement to prevent massive leakage of the bone cement that can damage the surrounding tissues.

The bag 30 can be made by folding a GBR membrane and suturing and/or gluing the edges of the membrane with a biocompatible glue. In another options the edges of the GBR membrane can be attached using biocompatible staples and/or tacks. The bag 30 can be already closed and filled with the bone augmenting material or can be empty and having one edge open to allow the surgeon to fill it with the bone augmenting material.

Although the present invention has been described and illustrated in the context of certain preferred embodiments, it will be understood that modifications may be made without departing from the spirit of the invention.

What is claimed is:

1. A device for treating a bone fracture comprising:
a sleeve having a stopping element, said sleeve being open on a first edge and open on a second edge, said sleeve being made from a guided tissue regeneration membrane, at least part of said stopping element being inside said sleeve, said stopping element being located at least 3 mm from said first edge and at least 3 mm from said second edge.

2. The device of claim 1, wherein said sleeve includes holes which are sized to allow the passage of fluids and nutrition and to prevent passage of cells.

3. The device of claim 1, wherein said stopping element being perforated with holes having diameter of more than 150 microns.

4. The device of claim 1, wherein said sleeve is made at least in part from bio-dissipative material.

5. The device of claim 1, wherein said sleeve includes a fixating element adjacent at least one of said edges, said fixating element selected from the group consisting of a suture, a wire, a band, a screw, a glue, a staple, a tack and any combination thereof.

6. The device of claim 1, wherein said device further includes a fixating plate and fixating screws.

7. The device of claim 1, wherein said stopping element is made from a bone augmenting material.

8. The device of claim 1, wherein said sleeve having a first stiffness and said stopping element having a second stiffness more than said first stiffness.

9. The device of claim 1, wherein said sleeve having a first mean time to resorption and said stopping element having a second mean time to resorption less than said first mean time to resorption.

10. The device of claim 1, wherein said device further includes a fixating plate and fixating screws, said stopping element being fixated to said fixating plate.

11. A kit for treating a bone fracture comprising:
a sleeve having a stopping element and a bone augmenting material, said sleeve being open on a first edge and open on a second edge, at least part of said stopping element being inside said sleeve, after the insertion of said bone augmenting material inside said sleeve said stopping element prevents the passage of at least part of said bone augmenting material through said stopping element.

12. The kit of claim 11, wherein said sleeve is made from a guided bone regeneration membrane.

13. The kit of claim 11, wherein said sleeve includes holes which are sized to allow the passage of fluids and nutrition and to prevent passage of cells.

14. The kit of claim 11, wherein said stopping element being perforated with holes having diameter of more than 150 microns.

15. The kit of claim 11, wherein said bone augmenting material includes particles having size of 200-500 microns.

16. The kit of claim 11, wherein said stopping element is made from a bone augmenting material.

17. The kit of claim 11, wherein said stopping element being located at least 3 mm from said first edge and at least 3 mm from said second edge.

18. The kit of claim 11, wherein said sleeve includes a fixating element adjacent at least one of said edges, said fixating element selected from the group consisting of a suture, a wire, a band, a screw, a glue, a staple, a tack and any combination thereof.

19. The kit of claim 11, wherein said device further includes a fixating plate and fixating screws, said stopping element being fixated to said fixating plate.

20. The kit of claim 11, wherein said sleeve having a first mean time to resorption and said stopping element having a second mean time to resorption less than said first mean time to resorption.
